(19) United States
(12) Patent Application Publication
(10) Pub. No.: US 2018/0169369 A1
(43) Pub. Date: Jun. 21, 2018

(54) OXYGEN REBREATHING APPARATUS AND METHOD FOR USING THE SAME

(71) Applicant: Joel Ironstone, Toronto (CA)

(72) Inventor: Joel Ironstone, Toronto (CA)

(21) Appl. No.: 15/739,058

(22) PCT Filed: Jul. 6, 2016
(86) PCT No.: PCT/IB2016/001153

§ 371 Date: Dec. 21, 2017

Related U.S. Application Data
(60) Provisional application No. 62/188,950, filed on Jul. 6, 2015.

(51) Int. Cl. A61M 16/08; A61M 16/00; A61M 16/10; A61M 16/22

(52) U.S. Cl. CPC A61M 16/0891; A61M 16/0045; A61M 16/12; A61M 16/22; A61M 16/101

(57) ABSTRACT

The present invention relates to a system and method for delivering oxygen for breathing at a higher concentration than found in normal air and therapeutic applications of such a system and method, which improves on the limitations of the prior art by reducing and practically eliminating the waste of oxygen gas. The main benefit of this approach is a reduction in the amount of oxygen required which may result in secondary benefits such as simplifying the logistics of acquiring and storing oxygen, reducing cost, improving system portability, and allowing for the use of low-flow sources such as an oxygen concentrator instead of only using high-flow sources such as tanks of compressed oxygen.

### RELATED APPLICATIONS

This application claims priority to U.S. Provisional Patent Application No. 62/188,950 entitled, “Oxygen Rebreathing Methods and Apparatus” filed on Jul. 6, 2015, which is hereby incorporated by reference in its entirety.

### TECHNICAL FIELD

The present disclosure relates to techniques for the delivery of oxygen for therapeutic or diagnostic breathing purposes. More specifically, this disclosure describes oxygen delivery apparatus for delivering oxygen to a patient for the treatment of cluster headaches and other conditions, and methods thereof.

### BACKGROUND

Cluster headaches involve repeated severe unilateral headaches lasting 15 to 180 minutes. These headaches often occur several times a day for several days, often at the same time of day. Typical cluster headache sufferers experience intervals in which headaches occur (a cluster) followed by headache-free intervals. A small proportion of sufferers are considered chronic and experience no headache-free intervals. Cluster headaches are currently managed with both preventative and abortive therapies. Preventative therapies are intended to reduce the frequency or severity of headaches, or terminate a cluster of headaches. Preventative therapies include anti-inflammatories and Verapamil. Abortive treatments are intended to stop an individual headache. Abortive treatments include Sumatriptan, high concentration (as much as 100%) oxygen delivered at high flows from an oxygen source, intranasal Zolmitriptan, and subcutaneous or intramuscular dihydroergotamine. Each of the pharmacological approaches has the potential for serious and life threatening side effects.

Inhaled oxygen has been shown in randomized controlled trials to be effective at aborting a cluster headache. The therapeutic response rate to oxygen in aborting cluster headaches is dependent on oxygen flow rates. It is generally believed that the higher the source flow rate, the more effective the treatment. Typically, flow rates from 7 liters per minute (LPM) to as high as 20 LPM are recommended for treating a cluster headache. However, since a cluster headache treatment with oxygen typically takes about 15 to 20 minutes, the treatment can require up to 400 liters for a single headache. This volume of oxygen requires a tank weighing over 12 pounds, which is also not conveniently transported or stored. Furthermore, since most cluster headache patients experience 2 to 8 headaches a day, treatment with high flow oxygen effectively confines the patient to a close proximity of a large tank or tanks, making many patients housebound and unable to work or enjoy other normal activities.

Several techniques have been described to deliver oxygen to patients. The source of oxygen is typically a pressurized oxygen tank, although other sources of high concentration oxygen might be used, including liquid oxygen or oxygen from a concentrator, all of which are comprehended by this disclosure as well. The most common method is by administration of a fixed flow of oxygen into a non-rebreathing facemask. With such an oxygen mask, oxygen is breathed in from the oxygen source, through a tube connecting the oxygen source to the mask, at a set rate. However, when the patient's ventilation rate exceeds the set oxygen flow rate, ambient air is drawn in through holes or a pressure-operated valve in the mask. This inhaled ambient air dilutes the concentration of oxygen delivered to the patient. Patients using oxygen masks exhale to the surrounding atmosphere. Therefore, some unbreathed oxygen from the source can be lost at the mask. Also, since a person does not absorb or metabolize all of the oxygen he or she inhales, and conventional oxygen masks do not separate the gases being exhaled, further unmetabolized oxygen present in the patient's exhaled breath goes unused and is wasted.

In some cases, a reservoir bag is added to the oxygen mask to collect gas delivered to the mask during exhalation. Yet, to prevent dilution of the delivered oxygen concentration with this configuration, the volumetric flow of oxygen must still be matched to the patient's minute ventilation. Otherwise, the excess flow demanded by the patient over the flow delivered from the source will be made up of room air, which comprises about 21% oxygen, substantially dilutes the high concentration oxygen delivered from the source.

A more recent advance is the use of a demand valve system which automatically matches oxygen supply from the source to the patient's minute ventilation. This allows the patient to increase their breathing rate and self-administer more therapeutic oxygen at a concentration consistently equal to the source concentration and more efficiently than use of a single, fixed, high-flow supply. Assuming the mask is well sealed and/or a sealed mouthpiece is used, no dilution of the source gas occurs because the demand valve automatically provides the flow demanded and no ambient air is drawn into the patient's lungs.

In order to deliver a concentration close to or at the concentration of the oxygen source, all of the prior techniques require an average volumetric flow of oxygen from the source at or above a patient's minute ventilation. This is typically in the range of 5 to 10 LPM, and in the case of cluster headache treatment, can be as high as 15 to 20 LPM. However, only a fraction of inhaled oxygen is consumed (metabolized) by the body, approximately 200 to 400 ml/min in the case of a resting adult. Consequently, most of the inspired oxygen is exhaled and lost to the surrounding atmosphere.

When minute ventilation and flow from the oxygen source are increased, the rate at which oxygen is wasted by being exhaled into the atmosphere also increases. When the patient's minute ventilation is increased without increasing the flow from the oxygen source, the supplied oxygen will be diluted with other air drawn into the system to make up the difference in volume of gas delivered. Maintaining high oxygen concentrations via the provision of high flows from the oxygen source means that large volumes of oxygen are required to treat a headache in current systems, which are inefficient and costlier than necessary to operate.

Techniques have been developed that recycle exhaled oxygen while removing the carbon dioxide produced by metabolism. Anesthesia ventilators use a carbon dioxide scrubber in the inspiratory limb of a ventilator circle system. In anesthesia ventilators, the patient exhales through a one-way valve into an expandable reservoir such as a bellows. The patient's exhaled gas is mixed with fresh gas and inhaled through a one-way valve and a carbon dioxide scrubber. Current anesthesia ventilators are not portable and weight upwards of 50 lbs. In addition, to enable ventilation, the expandable reservoir must be housed in a rigid body capable of being pressurized.

Diving rebreathing systems have been used for decades to reduce the compressed gas requirements (tanks carried) and extend the bottom time, relative to open breathing systems (i.e. SCUBA). In diving rebreather systems, exhaled gas is scrubbed of carbon dioxide and mixed with a fresh gas comprising oxygen, nitrox and/or inert diluents. The resulting gas mixture (either pure oxygen or a diluted mixture thereof) is cycled through (via one-way flapper valves) a counterlung (e.g. breathing bag, bellows, reservoir, etc.) from which the user inhales. Diving rebreathing systems are typically bulky (mixed gas) and sometimes comprise a rigid outer shell to protect the counterlung from environmental damage, such as from coral reefs, etc. Also, a potential safety hazard (e.g., hypoxia, oxygen toxicity, etc.) occurs if one or more of the compressed gas sources are exhausted.

Semi closed systems leak a modicum of gas and cycle (recirculate) the remainder. This limits how quickly very high inspired oxygen concentrations can be achieved. Specifically, a user's lungs begin with about 80% nitrogen, which upon exhalation in the counterlung, dilutes any oxygen delivered to the system.

As can be seen, conventional solutions to cluster headaches, including oxygen delivery techniques, are not ideal, cost-effective or convenient to use or administer. Prior art systems generally teach maximizing an oxygen flow rate from an oxygen source, without regard for the minute ventilation of a patient, and as such operate inefficiently and waste oxygen that could otherwise be useful. This disclosure seeks to address some or all of these issues.

### SUMMARY

In an aspect, the present disclosure recognizes the benefit of having the ability to achieve a high concentration of breathable oxygen using relatively low flows of oxygen for therapeutic purposes. While the descriptions below are mainly directed to treatment of cluster headaches, those skilled in the art can appreciate that these notions can be applied to treatment of headaches or other pains more generally. Such applications of the present examples are intended to cover all such useful applications.

This disclosure is not intended to provide an exclusive or exhaustive explanation of the invention. Further limitations and disadvantages of conventional and traditional approaches will become apparent to one of skill in the art, through comparison of such systems with some aspects of the present invention as set forth in the remainder of the present application with reference to the drawings.

The present invention relates to a system and method for delivering oxygen for breathing at a higher concentration than found in normal air and therapeutic applications of systems and methods thereof. The inventors have recognized that a factor in treating cluster headache and other conditions is the concentration of oxygen provided to the patient, which may not be identical to the rate of flow of oxygen from an oxygen source. In an example, the present invention relates to a method for treating cluster headaches and other conditions using a relatively lower flow of highly oxygenated gas relative to a patient's minute ventilation rate. Specifically, in rebreather configurations, selectively discarding expired gas for an initial duration of up to several minutes achieves high inspired oxygen concentrations in the closed circuit. Additionally, other aspects operate as disclosed, additionally in conjunction with scrubbing carbon dioxide from the exhaled gases from a patient to maximize the use of artificially delivered oxygen in the highly oxygenated gas.

The present invention achieves very high concentrations of oxygen while substantially reducing the waste of oxygen gas. One aspect of this approach is a reduction in the total amount of artificially stored and delivered oxygen required to achieve a therapeutic effect. This approach can result in secondary benefits, such as, simplifying the logistics of acquiring and storing oxygen, reducing cost, improving system portability, and allowing for the use of low-flow sources such as an oxygen concentrator instead of only using high-flow sources such as tanks of compressed oxygen.

According to one aspect, the present invention comprises an oxygen delivery system, sometimes referred to herein as a breathing circuit. The oxygen delivery system comprises a breathing interface, e.g., a mask or mouthpiece, which system facilitates a rebreathing of gas with a higher than normal oxygen concentration such that the oxygen in exhaled breath is recovered and reused for subsequent inhalation. As part of recycling the exhaled gas for rebreathing, carbon dioxide is removed from exhaled gas received back into the circuit.

According to one or more aspects, an oxygen source is used to fill the breathing circuit with a high concentration of oxygen gas for inhalation and to replace oxygen that has been consumed by the patient or lost through leaks thereby establishing and maintaining a desired oxygen concentration and/or volume in the circuit.

Therefore, a breathing apparatus is provided in various embodiments. A patient can be made to rebreathe or circulate inhaled and exhaled gases as desired, which is sometimes related to the concept of rebreathing herein and in the art. The present disclosure comprehends the use of the breathing apparatus and methods to include rebreathing as suitable, and thus the two terms appear in this disclosure, in which the appended claims are drafted to cover the general concept and design.

According one or more aspects, the rebreathing circuit comprises one or more valves or switches. In one aspect, a valve selectively exhausts exhaled gas volume or recycles exhaled gas volume for rebreathing by the patient. In one embodiment the valve is passive and set to open at a predetermined pressure. In another embodiment, the valve may be activated by mechanical switch or by an electrical switch, whereby it is in a first position for a period of time, and thereafter is switched to a second position.

According to another aspect of the present invention, a carbon dioxide scrubber may be disposed proximally to the breathing interface of the rebreather, or anywhere in the circuit where previously exhaled gas passes through it prior to returning to the patient for rebreathing.

According to another aspect, the pathway of the breathing circuit may be linear and/or substantially bidirectional.

According to yet another aspect, the pathway of the breathing circuit comprises one or more valves and/or switches, as well as inlets, outlets, and gas reservoirs.

According to another aspect, an oxygen source used to supply high concentration oxygen gas to the circuit is comprised of a compressed gas tank, a liquid oxygen tank, or an oxygen concentrator, or a combination thereof. According to some aspects, each of these sources may be connected to an appropriate regulator allowing the desired volumes to flow into the breathing circuit.

According to one aspect, a gas source may be connected to the breathing circuit by means of a demand valve (for example, a 302 OX-Series Inhalator Valve). According to another aspect, a gas source may be connected to the breathing circuit via a flow regulator. According to one aspect, the flow regulator has fully adjustable flow settings. According to other aspects, a regulator with only a limited number of settings, such as, off, low-flow, or high-flow is used.

According to some aspects, the present invention is a method for treating cluster headaches by providing oxygen gas to a rebreathing circuit and rebreathing high concentration oxygen gas while scrubbing CO2 from recaptured exhaled breath.

According to one aspect, the method comprises venting some exhaled breaths out from the rebreathing circuit while recycling a substantial amount of exhaled breaths back to the circuit for rebreathing.

According to one aspect, a method comprises providing a high flow of source gas (for example, high concentration oxygen) for a first phase. According to some aspects, the method further comprises providing a lower flow of source gas for a subsequent phase.

According to one or more aspects, a method comprises providing oxygen gas to the circuit by means of a demand valve and/or flow regulator. Specifically, a first stage regulator is attached to the valve of a gas source (e.g., gas bottle, tank, etc.). The first stage converts the high pressure of the gas source to an intermediate pressure. According to one aspect, an intermediate pressure hose couples the first stage regulator to a second stage regulator. The second stage is a demand type gas delivery device which converts intermediate pressure to the ambient pressure of the reservoir and rebreather circuit.

According to some aspects, the method for treating pain, headaches, or a cluster headache and/or other condition comprises using a low flow of gas with a high concentration of oxygen from a gas source. According to another aspect of the invention, the method comprises treating a cluster headache or other headache condition using a series of steps. The steps include using a high flow of gas with a high concentration of oxygen from a gas source during a first phase (referred hereinafter as the flush phase) followed by a low flow of gas with a high concentration of oxygen from a gas source during a second phase (referred hereinafter as the maintenance phase). The flow in the flush phase is adapted to raise the concentration of oxygen in the patient's lung rapidly and the flow in the maintenance phase is adapted to maintain the concentration of oxygen in the patient's lung using a minimal flow.

One particular inventive method is directed to treating pain, e.g., headaches such as cluster headaches, in a patient, comprising capturing exhaled gas from the patient, and scrubbing at least part of the exhaled gas using a carbon dioxide scrubber; and delivering to the patient a mixture of scrubbed exhaled gas as well as highly oxygenated gas from a source of highly oxygenated gas, wherein said highly oxygenated gas is provided from said source at a rate below the patient's minute ventilation rate.

Another particular inventive method is directed to treating pain in a patient, using a breathing apparatus, the method comprising coupling the breathing apparatus to a source of highly oxygenated gas, said highly oxygenated gas having an oxygen concentration greater than that of a naturally occurring environment; providing a breathing interface of said breathing apparatus to the patient to inhale from and to exhale into; in a first (flushing) mode of operation: (a) discharging exhaled gas from the patient to said environment, and (b) providing the patient for inhalation said highly oxygenated gas, from said source, via said breathing apparatus; and in a second (maintenance) mode of operation: (a) receiving the patient's exhaled gas in said breathing apparatus, (b) scrubbing at least part of said patient's exhaled gas with a carbon dioxide scrubber in said breathing apparatus, and (c) providing the patient for inhalation at least part of the scrubbed exhaled gas.

Yet another inventive method is directed to for treating pain in a patient, using a breathing apparatus that the patient inhales from and exhales to, the method comprising capturing exhaled gas from the patient, and scrubbing at least part of the exhaled gas using a carbon dioxide scrubber in said breathing apparatus; and delivering to the patient a mixture of scrubbed exhaled gas as well as highly oxygenated gas from a gas source, said highly oxygenated gas having a concentration of oxygen greater than an atmospheric concentration of oxygen, and said highly oxygenated gas being delivered at a flow rate less than a minute ventilation rate of the patient.

Still another inventive aspect is directed to breathing apparatus, comprising a breathing interface from which a patient inhales and into which the patient exhales; a gas source containing a highly oxygenated gas having a greater concentration of oxygen than an atmospheric concentration of oxygen; a supply gas inlet, placing said apparatus in fluid communication with said gas source, and providing a flow of said highly oxygenated gas to the patient; a gas flow regulator that regulates a rate of supply of said highly oxygenated gas such that said rate of supply is at least sometimes less than a minute ventilation rate of said patient during use; and a carbon dioxide scrubber that receives exhaled gas from the patient; wherein the highly oxygenated gas provided through the gas inlet, and the exhaled gas scrubbed by the carbon dioxide scrubber are provided through said breathing apparatus, via the breathing interface, to the patient inhaling therefrom.

And yet another inventive embodiment is directed to a method of easing pain in a patient using a breathing apparatus coupled to a highly oxygenated gas source and having a gas reservoir and a carbon dioxide scrubber, the method comprising capturing exhaled gas from said patient in said reservoir; removing carbon dioxide from said exhaled gas using said carbon dioxide scrubber; providing the patient, for inhalation through the breathing apparatus, at least a portion of the captured exhaled gas and at least a portion of the highly oxygenated gas from the source of highly oxygenated gas; and continuing to provide the aforementioned gases to the patient, for inhalation, until a desired reduction in said pain is achieved.

The drawings show exemplary breathing circuits. Variations of these circuits, for example, changing the positions of, adding, or removing certain elements from the circuits are not beyond the scope of the present invention. The illustrated breathing circuits are intended to be complementary to the support found in the detailed description.

### BRIEF DESCRIPTION OF THE DRAWINGS

For a fuller understanding of the nature and advantages of the present invention, reference is made to the following detailed description of preferred embodiments and in connection with the accompanying drawings, in which:

FIG. 1 shows an exemplary linear rebreathing circuit, in accordance with some embodiments of the disclosure provided herein;

FIG. 2 shows an exemplary linear rebreather circuit comprising inspiratory and expiratory relief valves, in accordance with some embodiments of the disclosure provided herein;

FIG. 3 shows an exemplary rebreathing circuit comprising a constant flow gas delivery system and a switch to direct exhaled gas, wherein when the switch is in one setting, gas exhaled into the breathing interface will be vented out of the circuit through the expiratory relief valve and, when the switch is in another setting, exhaled gas is directed to the counterlung, in accordance with some embodiments of the disclosure provided herein; and

FIG. 4 shows an exemplary rebreathing circuit comprising a demand valve, which controls flow of source gas into the circuit using a switch to direct exhaled gas, wherein when the switch is in one setting, gas exhaled into the breathing interface will be vented out of the circuit through the expiratory relief valve and, when the switch is in another setting, gas can flow to the counterlung, in accordance with some embodiments of the disclosure provided herein.

### DETAILED DESCRIPTION

The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Other objects, advantages and novel features of the disclosure are set forth in the proceeding in view of the drawings where applicable.

The therapeutic applications of the present system and methods can vary. In an embodiment, these can be applied to treatment of cluster headaches. In another embodiment, these can be applied to treatment of headaches generally. In yet another embodiment, these can be applied to treatment of pain generally in a patient. Those skilled in the art will best appreciate the use of the present systems and methods and the applications thereof in their fields of expertise.

As mentioned, the invention is directed in some aspects to treating pain, headaches, or cluster headaches in a patient using a breathing apparatus as described below. The present apparatuses can be characterized as breathing or rebreathing as appropriate in a given context, but no literal distinctions should be applied to these terms unless provided by the present disclosure.

A patient is of a known or measurable minute ventilation rate. The patient is supplied with a rebreathing apparatus having a breathing interface that can be secured or manually held to the patient's mouth and/or nose to breathe into and out of. In an aspect, the patient can be made to exclusively breathe in/out of the breathing interface to control the gas content received by the patient during a treatment.

A source of highly oxygenated gas, having an oxygen concentration well above an atmospheric oxygen concentration, is coupled to said rebreathing apparatus. In some embodiments, the highly oxygenated gas comprises nearly pure oxygen gas. In other embodiments, the highly oxygenated gas comprises a concentration of oxygen being at least twice that of atmospheric air.

A gas flow regulator may control the volumetric flow of highly oxygenated gas to at least a portion of the rebreathing apparatus in some embodiments. In other embodiments, a flow measuring device such as a rotary, Venturi, or similar device is used to measure the flow of gases in the system. In addition, the rebreathing apparatus captures exhaled gas from the patient, via the breathing interface, which in some modes of operation is scrubbed in the apparatus by a carbon dioxide (CO2) scrubber. The patient is thus made to rebreathe a mixture of scrubbed exhaled gas and the provided highly oxygenated gas. A demand valve may be employed in some embodiments to regulate or provide a supply of highly oxygenated gas to the patient at a desired rate, for example at a rate measured relative to the patient's minute ventilation rate.

As the exhaled gas may contain a rich amount of un-absorbed oxygen that is released with the exhaled gas, this unused exhaled oxygen can be re-breathed and exploited for use herein. One result is that the patient's condition is treated by the elevated oxygen delivery to the patient. Another result is that the source of highly oxygenated gas can be more effectively used, e.g., requiring a smaller container of said highly oxygenated gas, because the oxygen is not wastefully exhaled to the ambient environment as with some other systems. Therefore, a more cost-efficient, compact and portable system can be made and used by patients.

An aspect of the present invention is directed to a method and apparatus for the application of oxygen therapy. More specifically, the present invention discloses the method of using an oxygen rebreather for delivering high concentrations of oxygen to a patient for the treatment of cluster headaches and other conditions while minimizing waste by recycling expired gas. The present invention also discloses novel oxygen rebreather embodiments which are particularly well-suited for carrying out said method.

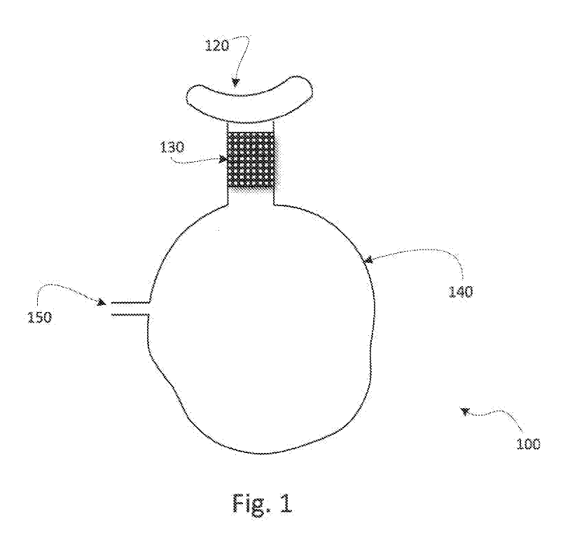

Turning to FIG. 1, an exemplary linear breathing circuit 100 delivers gas through a breathing interface 120 for inhalation. In one embodiment, the breathing interface 120 is a dental mouthpiece held in place with a patient's teeth or gums. In another embodiment, the breathing interface 120 is a mask which circumscribes the mouth and/or nose. In other embodiments, the breathing interface 120 is a full-face mask, such as one used in SCBA or firefighting equipment. However, any suitable substantially sealable interface is not beyond the scope of the invention.

In practice, exhaled gas is received back into the breathing circuit 100 through the breathing interface 120. The gas traverses the circuit to (or is held, at least in part, by) a counterlung 140 (sometimes referred to as reservoir) which forms part of the circuit until inhaled again in later breaths. This allows the oxygen which remains in the exhaled breath to be rebreathed, instead of releasing it into the surrounding air and requiring fresh oxygen for the complete volume of each inhalation. In one or more embodiments, the counterlung 140 is bellows or breathing bag. However, any substantially pliable volume of suitable material can be used such that it maintains a minimal pressure difference between the interior of the breathing circuit and ambient atmosphere to facilitate respiration.

As gas moves through the breathing circuit 100, gas passes through a carbon dioxide scrubber 130 which is generally disposed between the breathing interface 120 and counterlung 140. A carbon dioxide scrubber is a device which absorbs carbon dioxide (CO2). It is known in several arts to treat exhaust gases from industrial plants or from exhaled air in life support systems such as rebreathers, anesthesia machines, or in spacecraft, submersible craft or airtight chambers. CO2 scrubbers typically comprise tightly packed granular soda lime, sodium hydroxide, potassium hydroxide, and/or lithium hydroxide which able to remove carbon dioxide by chemically reacting with it. However, any suitable strong base or CO2 reactant is not beyond the scope of the present invention.

CO2 scrubber 130 removes carbon dioxide from the gas mixture so that the concentration of carbon dioxide remains at a low level similar to atmospheric air. Increasing CO2 partial pressures give rises to difficult breathing and an increased desire to breath leading to hyperventilation. Increased CO2 level can also lead to cerebral vasodilation and counteract the desired vasoconstriction effect of the oxygen. To this end, the gas in the breathing circuit 100 that is rebreathed must pass through the scrubber 130 at least once but may pass through the scrubber 130 more than once or pass through a plurality of scrubbers. The scrubber 130 is usually located in between the breathing interface 120 and the counterlung 140 or in between the breathing interface 120 and some other circuit volume that acts as a counterlung. However, the scrubber 130 could be located elsewhere in the circuit such as embedded within the counterlung or placed anywhere in the circuit where the gas being rebreathed will pass through it.

The size and shape of the scrubber 130 can be optimized to balance between the scrubbing efficiency, airflow resistance introduced by the scrubber, reactant longevity, and the volumetric capacity of the scrubber. The location of the scrubber 130 in the circuit can also be optimized so that the resistance it creates to airflow will not be between circuit elements where that resistance would be problematic or so that the resistance it creates to airflow will be between circuit elements where that resistance might be beneficial. In one or more embodiments, the CO2 scrubber 130 is disposed in the exhalation pathway, as it is easier for the lungs to exhale with increased gas resistance relative to inhalation with increased gas resistance.

Oxygen is also added to the gas mixture in the circuit as needed through an input port 150, both to replace oxygen that has been consumed so as to maintain the oxygen concentration and/or volume and to increase the volume and/or concentration of oxygen available for inhalation from the circuit.

In some embodiment, it is advantageous for the flow of oxygen into the circuit to be higher than the rate of oxygen consumption when the circuit is initially being filled with said highly oxygenated gas. The flow rate is regulated by a gas flow regulator. In some aspects, or in a temporary or optional mode of operation, the flow rate may be higher than the ventilation rate of the user so that the entire ventilation can be drawn from high concentration oxygen gas provided while still having additional flow for filling of the circuit 100 and counterlung 140.

The flow rate of the highly oxygenated gas is controllable and variable, for example, if the circuit needs to be filled very quickly or filled while already in use. In other cases, a lower initial flow rate may be acceptable, for example if the circuit 100 is being pre-filled with oxygen before use in breathing. The oxygen input port 150 may be located anywhere in the circuit, for example, coupled to the counterlung 140 so that oxygen fed into the circuit will enter the counterlung 140 directly, or as part of the breathing interface 120 so that this oxygen is more readily inhaled.

The gas storage capacity of the breathing circuit 100 should be equal to or greater than the volume of gas inhaled with each breath, so that all the gas required for inhalation can be drawn from the circuit 100. Accordingly, the breathing circuit 100 comprises some type of dynamic volume to accommodate each breath, at least in part.

In one or more embodiments, this dynamic volume (reservoir or counterlung 140) is a bellows or breathing bag or other suitable device known in the art, in order to provide sufficient gas storage volume. This counterlung 140 may be a dynamic rigid structure (e.g., accordion bellows, etc.), or may be collapsed, folder-like, rolled up, or compressed in some other way when not in use. It is preferable that the counterlung 140 can easily collapse in order to avoid significant negative pressure during inhalation and also to enable hyperventilation which is facilitated by a counterlung 140 that can collapse easily.

In some embodiments, the counterlung 140 may also be designed to expand and/or contract automatically as gas enters and leaves the circuit 100, either as a result of the elasticity of the counterlung 140 or responding in a different way to changes in gas pressure and/or volume in the circuit. If the gas is stored in the main circuit volume and not in a separately identifiable counterlung, then the terms counterlung and circuit may be used somewhat interchangeably.

The high concentration oxygen source may be a cylinder of compressed gas or optionally an oxygen concentrator or liquid oxygen container, or any combination of the above, which are coupled to the intake port 150. High concentration oxygen cylinders may be steel or aluminum tanks, which are well known the art. Optionally, the source gas can be stored in fiberglass canister such as those used in SCBA systems, which are known in the firefighting and other arts. These typically deliver gas using first and second stage regulators which reduce the pressure to an intermediate pressure and mechanically deliver gas on demand. In other embodiments, oxygen is delivered to the intake port using a low pressure oxygen concentrator which is known in the medical arts.

In some embodiments in greater detail later in the disclosure, the oxygen source may be coupled to the intake port 150 using a demand valve. A demand valve automatically opens and provides oxygen to the circuit when a negative pressure is applied to the breathing circuit 100, which occurs when the inspired volume of gas begins to exceed the amount of gas available in the circuit.

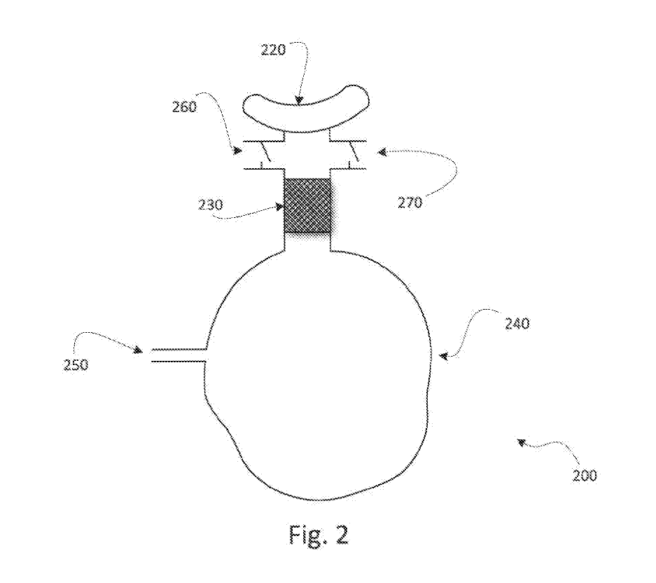

FIG. 2 shows an exemplary linear rebreather circuit comprising inspiratory and expiratory relief valves, in accordance with some embodiments of the disclosure provided herein. Rebreather circuit 200 comprises an inspiratory relief valve 260 and an overfill relief valve 270. Inspiratory relief valve 260 allows additional air into the circuit when the gas pressure inside the circuit drops below the air pressure outside the circuit by an amount greater than the cracking pressure of the inspiratory relief valve 260, in case there is not enough gas in the circuit to supply the entire gas volume required for inhalation.

Such a situation can arise when the user attempts to inhale a larger volume of gas than is contained in the circuit causing a drop in pressure inside the circuit. This inspiratory valve 260 may be part of or close to the breathing interface 220, in order to minimize rebreathed unscrubbed gas and minimize the intervening volume which could become clogged or blocked and obstruct inhalation. However, there may be larger dynamic pressure changes close to the breathing interface 220 during inhalation and exhalation. Therefore, it may be preferable for this valve to be placed more distal to the interface, for example, as part of or close to the scrubber 230 or reservoir 240. If the oxygen source is coupled to the oxygen intake port 250 via a demand regulator, relief valve 260 would not be used as it might preferentially allow room air in instead of source oxygen when the pressure in the breathing circuit 200 became negative.

Overfill relief valve 270 prevents the gas pressure in the circuit from getting too high, a situation which could cause barotrauma (embolism, pneumothorax, etc.), damage to the circuit, and/or make breathing with the circuit, especially exhaling into the circuit, difficult. The overfill relief valve allows gas out of the circuit if the gas pressure inside the circuit is greater than the gas pressure outside the circuit by an amount greater than the cracking pressure of the overfill relief valve 270. The overfill relief valve may be placed anywhere in the circuit, for example, as part of or close to the counterlung 240 or as part of or close to the breathing interface 220.

In the embodiments shown FIGS. 1 and 2, the gas delivered to the breathing interface 220 through the breathing circuit 200 for inhalation and gas received back into the breathing circuit through the breathing interface from exhalation travels through the circuit along substantially the same path. Having the same path for gas moving in both directions may result in a smaller breathing circuit which could have the advantages of less dead space, lower cost, greater portability, and/or easier storage. In the instant embodiments, gas has a minimum number of valves to pass through which reduces the internal circuit resistance to airflow and therefore makes breathing with the circuit easier.

Prior to use the breathing circuit 200 usually contains ambient air which has diffused therein. Oxygen is then fed into the circuit 200 and the user begins breathing using the circuit. It is possible for the user to begin breathing using the circuit either before, during, or after it is filled with the desired volume and concentration of oxygen gas. However, it is preferable for the circuit to be filled with the desired volume and concentration of oxygen before the user begins breathing with the circuit so that there is enough gas for the user to breathe. It is also desirable to preclude atmospheric air which has lower oxygen concentration being drawn through the inspiratory relief valve 260, ensuring proper oxygen concentration is delivered from the first breath.

In some situations, when the circuit 200 is being filled with the desired volume and concentration of oxygen before the user begins breathing with the circuit 200, it may be beneficial to seal or close the breathing interface 220 in order to prevent oxygen from escaping or normal air from entering through the breathing interface 220. However, in some circuit configuration embodiments, there are already other valves which will serve that purpose. For example, a one way valve from the counterlung to the breathing interface can prevent gas from leaving the reservoir through the breathing interface until the gas pressure in the counterlung is high enough, relative to the gas pressure at the breathing interface, to overcome the cracking pressure of the one way valve. Such a valve will also prevent gas from entering the counterlung from the breathing interface.

The functions of the breathing circuits 100, 200 are as follows. Normal (atmospheric) air residing in the circuit prior to use is made up of approximately 78% nitrogen and 21% oxygen, in addition to small amounts of argon, carbon dioxide and other trace gasses. As high concentration oxygen gas is fed into the breathing circuits 100, 200, the concentration of oxygen increases in the rebreather gas from relatively low while the present atmospheric air is forced out.

In embodiments in which the circuit comprises a collapsible counterlung 240, the counterlung 240 will be inflated by the additional volume of gas, either as soon as oxygen begins to be added or only after the added oxygen has forced some or all of the normal air out of the smaller circuit volume. In order to minimize the volume of air that needs to be washed out of the circuit, it is preferable for the counterlung 240 to be collapsed until it is needed to hold the gas fed into the circuit 200. Once the circuit 200 reaches its full volume, continuing to add oxygen will also increase the pressure in the circuit and continue to force out some, if not most, of the remaining normal air, thereby further increasing the oxygen concentration in the circuit.

When the user begins inhaling from and exhaling into the circuit 200, the gas mixture in the circuit 200 will be made up of air remaining in the circuit from prior to use, oxygen fed into the circuit, and gas from the lungs of the user prior to use. In one aspect of the invention, the objective of the method is to treat cluster headache or other conditions by the provision of high concentration oxygen to a patient. It is possible to operate the circuits 100, 200 described above with a relatively reduced or low flow of highly oxygenated gas (e.g., medical grade oxygen supply), provided at a rate lower than a patient's minute ventilation rate, thereby reducing the oxygen supply requirements. However, while circuits 100 and 200 are effective in minimizing the flow and volume requirement of oxygen for treatment of a cluster headache, this minimization of volume means it may take longer to get to a concentration effective to treat the headache.

The gas in the patient's lungs and all of the gas in the circuit are in fluid communication. From the perspective of oxygen concentration in the lungs and in the circuit, all of the volume of the circuit and lung can be thought of as a single volume. Oxygen added to the circuit will increase the oxygen concentration in the circuit 200 and lungs as it slowly washes out the air present in the total volume. The concentration increase in oxygen generally follows the form [O2]=S(1−e^{-kt}) where S is the concentration of oxygen in the source gas, k is a constant inversely related to the overall volume in the circuit 200 and lungs and proportional to the flow rate of high concentration oxygen gas into the circuit, and t is time.

For a given lung and circuit volume, if the gas flow into the circuit is high, the faster the oxygen in the lungs and circuit approach the concentration in the source gas. Carbon dioxide from the lungs of the user will be filtered out by the scrubber 230. To those skilled in the art, it will be understood that increasing the flow rate of source oxygen into the breathing circuit 200 will speed up the rise in concentration in the circuit and the patient's lungs. However, doing so will also require a greater volume of oxygen.

It is desirable to minimize the volume of oxygen required to treat a cluster headache so as to enable use of a small portable oxygen tank, while at the same time provide a rapid rise in oxygen concentration in the patient's lungs, and by extension, in the breathing circuit 200. The inventors have discovered that it is possible to achieve these very high concentrations of oxygen in the gas mixture in the circuit and to increase the oxygen concentration in the circuit quickly, by providing a breathing circuit that is capable of operating in two modes: a high flow flush mode and a low flow maintenance mode.

In the flush mode low oxygen concentration exhaled air is vented to atmosphere while high flow high oxygen concentration enters the circuit. Once high oxygen concentration in the circuit is achieved, in the maintenance mode, exhaled gas is recycled through the scrubber. It is desirable to flush the initial exhaled breaths into atmosphere because they contain very low concentrations of oxygen, essentially remnants of the air that was in the lungs prior to use of the circuit.

Once a high concentration of oxygen is achieved in the lungs, recycling exhaled gas does not substantially decrease the concentration in the circuit when mixed with new highly oxygenated gas, thus saving it by recycling is highly efficient. It is beneficial for the circuit 200 volume (including the counterlung 230) to be large enough that the user can inhale several breaths fully while venting the exhaled breath so that no additional air will need to be drawn in through the inspiratory relief valve 260 and dilute the high concentration oxygen gas.

Once the circuit 200 and the user's lungs have been cleared of high nitrogen concentration air and replaced with high concentration oxygen gas fed into the circuit 200 through oxygen inlet 250 and inhaled, the exhaled breath can once again be received into the breathing circuit 200 and a much higher oxygen concentration can be achieved in the gas mixture in the circuit 200. This result can also be achieved with a lower flow and volume of gas from the oxygen source. Note that for the purpose of these descriptions, gas which is exhaled into the breathing interface 220 but then vented out of the circuit 200 instead of being received into the main volume of the circuit is not considered as having been received into the circuit, even though the breathing interface 220 is a part of the circuit 200 itself.

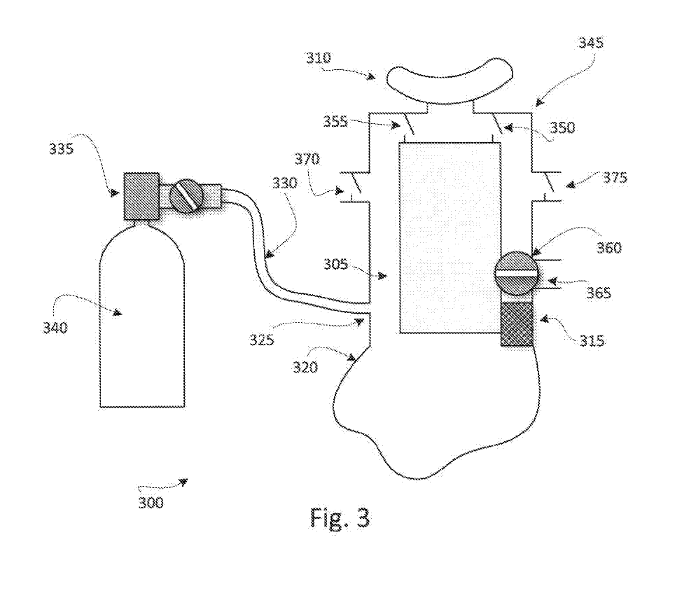

FIG. 3 shows an exemplary rebreathing circuit comprising a constant flow gas delivery system and two pathways for conducting exhaled gas received back into the circuit, a switch to select one of the two pathways, in accordance with some embodiments of the disclosure provided herein. Breathing circuit 300 comprises a breathing interface 310 in fluid communication with an exhalation tube 345 which is in turn connected to a switching valve 360.

In one or more embodiments, switching valve 360 is ball valve having the capacity to switch between a plurality of gas pathways. In other embodiments, switching valve 360 is a 3-way stopcock. Switching valve 360 accepts gas coming from the breathing interface 310 down the exhalation tube 345 and selectively routes it either out an exhaust port 365 which vents to atmosphere or onward in the circuit to a CO2 scrubber 315. The CO2 scrubber 315 is further fluidly connected to a gas reservoir 320 which collects exhaled gas for rebreathing when the switching valve 360 directs gas through the scrubber 315.

Reservoir 320 is fluidly connected to an oxygen port 325 which accepts oxygen via a conduit 330 from an oxygen source 340 which contains high concentration oxygen. Flow of the oxygen from the source 340 to the port is controlled by flow controller 335. In the present embodiment, flow controller 335 is a gas tank oxygen flow regulator as is common in the art. An inspiratory tube 305 connects the reservoir back to the breathing interface 310. An inspiratory one-way valve 355 is disposed somewhere along the inspiratory tube 305. An expiratory one-way valve 350 is disposed somewhere along the expiratory tube 345 between the breathing interface 310 and the switching valve 360.

In an exemplary embodiment, the function of the breathing circuit 300 is as follows. For a first time period called the flush phase, the switching valve 360 is set to direct gas to the exhaust port 365. The flow controller 335 is set to a high flow rate. This can be greater than 7 LPM and optionally as high as 30 LPM or higher. The limit to how high this should be set is governed by oxygen use and the patient's minute ventilation rate. The higher this is set, the greater the oxygen use during the flush phase, but the faster the rise in concentration will be. In this example, any flow above the patient's minute ventilation rate may be wasted. Oxygen enters the circuit 300 by way of conduit 330 and port 325. Port 325 is shown disposed along the inspiratory limb 305 but may be anywhere along the gas path of the circuit 300. Oxygen fills the reservoir 320 and the patient inspires gas from the reservoir 320 along the inspiratory limb 305 via the one-way valve 355 and through the breathing interface 310. One-way valve 350 ensures that air cannot be inspired from the exhaust port 365. The patient exhales back through the patient interface 310 and exhaled gas is directed through the one-way valve 350 down the exhalation tube 345. Switching valve 360 directs the exhaled gas out the exhaust port 365.

The time to switch modes from flush phase to maintenance phase may be determined by the elapsing of a predetermined time, a predetermined number of breaths, or a predetermined concentration of oxygen having been achieved in exhaled breath as measured by a rapid oxygen analyzer (not shown) as is well known in the art. To switch modes, the switching valve 360 is turned to block the exhaust port 365 and direct exhaled gas towards to reservoir 320 via the scrubber 315. The flow controller 335 is turned to a low flow setting. Preferably, this flow may be less than the patient's minute ventilation rate, for example, less than 2 LPM, or optionally less than 1 LPM or even 0.5 LPM. A setting of 1 LPM (generically used here as an example) is typically sufficient to maintain the high oxygen concentration for most human subjects at rest whose oxygen consumption would typically be less than 0.5 LPM. Those skilled in the art will appreciate variations to the above examples, which can depend on the patient in question, e.g., the patient's weight, age, gender, physical condition, natural metabolic rate, and other factors.

In the maintenance phase or mode of operation, exhaled gas passes through the scrubber 320 at least once prior to being rebreathed. Inspiratory relief valve 370 opens a lower negative pressure than is required to inhale from the reservoir 320 in order to provide gas if the breathing rate exceeds the flow rate of gas from the source. Expiratory relief valve 375 allows gas to escape if the system ever becomes over pressurized. Relief valve 375 has a cracking pressure that prevents gas from being inadvertently vented to atmosphere during the maintenance mode, except in an overpressure condition, e.g., one that might cause barotrauma to the patient. An exemplary cracking pressure may be set to at least or about 10 cm H2O. This setting can be fixed and based on a factory manufacturing specification, or it may be adjustable using a set point adjustment feature of the relief valve 375. It can be appreciated that the order or position of some of the elements described herein may be changed without affecting the function of the circuit 300 and that many other embodiments of the invention are possible without departing from the scope of the disclosed invention.

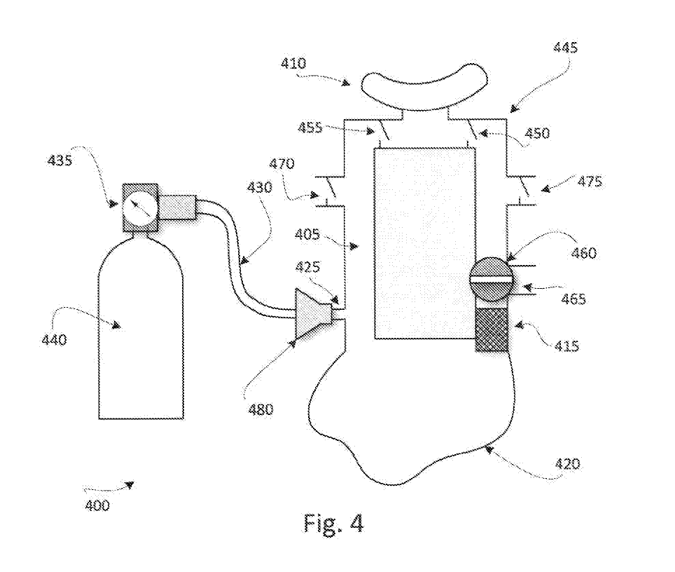

FIG. 4 shows an exemplary rebreathing circuit 400 comprising a demand valve 480 and two pathways 405, 445 between the breathing interface 410 and the reservoir 420, and a switch 460 to select between directing exhaled gas received into the circuit 400 either out into atmosphere, or further into a reservoir 420, in accordance with some embodiments of the disclosure provided herein. Notably distinct from FIG. 3, rebreather circuit 400 comprises a demand valve 480 interposed between oxygen port 425 and conduit 430. Pressure in oxygen source 440 which contains high concentration oxygen is regulated to a lower pressure acceptable as input to the demand valve 480 by pressure regulator 435, which may for example regulate pressure to approximately 50 psi. In this embodiment, no changes are required to be made by the user the change the flow rates into the circuit.

Similar to the operation of the circuit 300, for a first time period, called the Flush phase, the switching valve 460 is set to direct gas to the exhaust port 465. There is no need to prefill the reservoir 420. Inhalation from the circuit will cause the demand valve 480 to open allowing oxygen to flow into the circuit 400 and hence into the patient/user. The flow of the demand regulator 480 is automatically matched to the flow demanded by the patient, making the system extremely efficient. In contrast, for example, in the fixed flow embodiment described in FIG. 3, the source oxygen flow into the circuit 300 might be set at 15 LPM. However, some patient's undergoing cluster headaches desiring to breathe 20 LPM on average due to agitation or a desire for faster relief. Such a patient would eventually see the reservoir 320 depleted and on subsequent breaths, negative pressure would open the relief valve 370 diluting oxygen concentration. Conversely, a patient breathing only 12 LPM during flush mode would be wasting 3 LPM if flow were set at 15 LPM.

To switch modes from flush mode to maintenance mode, a mode switching valve 460 is turned to block the exhaust port 465 and direct exhaled gas towards to reservoir 420 via the scrubber 415. No change is required to the source gas flow. During the first breaths in maintenance mode, the patient will breathe in volume from the reservoir 420 and his tissues will consume some oxygen, typically at a rate on the order of about 300 mL/minute. Since the demand regulator valve 480 will not open until small negative pressure is reached in the circuit 400, with each breath the reservoir 420 will become slightly depleted until it is almost empty. For subsequent breaths, the demand valve 480 will open allowing a small amount of oxygen into the circuit 400. This occurs for a portion of the breath while the patient is inspiring and the reservoir 420 becomes empty.

In the present embodiment, the amount of oxygen provided by the opening of the demand valve 480 just at the end of each breath, effectively provides the low flow rate into the system required to maintain oxygen concentration during the Maintenance phase. Since demand valve 480 provides a flow rate tailor matched to the oxygen consumption of the patient during maintenance phase, the present invention is more efficient than circuits where the oxygen flow rate entering the circuit is fixed. For example, in the fixed flow circuit described in FIG. 3, setting the flow at 1 LPM would be sufficient to meet demands of most patients, since oxygen consumption by tissues is can be 250-1000 ml/min. depending on the agitation and physical movement of the patient. However, this would waste 0.75 LPM for a patient whose tissue consumption was at the low end of the range and 0.5 LPM for a patient at the high end. In the present embodiment, each patient during Flush phase, has the flow rate into the circuit 480 tailor matched to the consumption requirements of the patient by the function of the demand valve 480 described above.

In any of the embodiments described above, it may be desirable to include one or more of the following additional elements: one or more gas flow sensors to measure gas flow through one or more parts of the circuit; one or more gas sensors (e.g., O2 lambda, etc.) to measure the concentrations of one or more gasses in the circuit; one or more sensors or timers to measure the status or usage of the scrubber and indicate when the scrubber is reaching the end of its effective use; one or more devices added to one or more of the relief valves that will make a sound or give some other indication to the user when gas is flowing through the relief valve; an air filter, humidifier, dehumidifier, desiccant material, heater, cooler, purifier, or other device for conditioning the gas in the circuit.

Additional ports on one or more components of the breathing circuit may also be needed to enable coupling of such additional elements to the circuit. In any of the embodiments described above it may also be desirable to design the circuit with one or more of the following features. For example, the gas counterlung or scrubber may be removable from the circuit and reattached or replaced. The breathing apparatus and carbon dioxide scrubber may be combined in a single counterlung-and-scrubber module which may be separated from the rest of the circuit and reconnected or replaced. The scrubber material may be embedded in the counterlung instead of being a distinct component. The size of the carbon dioxide scrubber may be matched to the amount of CO2 expected to pass through the scrubber during the expected time of use of the circuit during a single session of use.

The efficient method of therapeutically delivering gas with a higher than normal concentration of oxygen disclosed in the present invention can be performed using any of the breathing circuits described above or other variations of those circuits. The rebreathing circuits presented in the present disclosure have advantages over other circuits known in the art as already discussed (more efficient use of oxygen, higher oxygen concentrations, faster increase in oxygen concentration, smaller size, etc.).

In some aspects, the present system and method allow for delivering breathable high concentration oxygen gas and achieving the desired result of inducing vasoconstriction in response to elevated levels of blood oxygen. This response may be beneficial in treating neurovascular conditions including pain, headache, cluster headache, and migraine headache.

The method of delivering and rebreathing gasses described herein might also be used for breathing or rebreathing of other gas mixtures, or in other applications besides treating the conditions described. When the gas mixture being delivered and rebreathed contains oxygen concentrations higher than normal air, the range of concentrations can be anywhere from slightly or moderately higher concentration than normal air up to substantially pure oxygen. However, the benefits of rebreathing the gas mixture and recycling the unused oxygen increase as the desired oxygen concentration increases.

The method of oxygen delivery using a breathing circuit comprises delivering oxygen to a breathing circuit through a gas intake port for inhalation through a breathing interface such as a mask or mouthpiece; receiving exhaled gas back into the breathing circuit through the breathing interface; removing carbon dioxide from the gas in the breathing circuit using a carbon dioxide scrubber; and storing oxygen and/or exhaled gas in a counterlung in the breathing circuit. When using one of the breathing circuits described above the method is also generally such that gas moving from the breathing interface to the counterlung follows substantially the same pathway as gas moving from the reservoir to the breathing interface. However, this method also includes the use of a rebreathing circuit with a circular flow configuration.

The method may optionally further comprise one or more of: allowing oxygen from the oxygen source into the circuit through a demand valve when inhalation draws more gas than available inside the breathing circuit; allowing air into the circuit through an inspiratory relief valve when inhalation draws more gas than available inside the breathing circuit; allowing air out of the circuit through an expiratory relief valve when the volume and/or pressure of gas inside the breathing circuit is too high; switching between a first state in which the exhaled gas received back into the circuit is directed into the counterlung and a second state in which the exhaled gas is vented out of the breathing circuit; measuring the pressure, flow, and/or concentration of gasses in the circuit; measuring and indicating the status of the scrubber and its remaining scrubbing capacity; adding to one or more of the relief valves a device that will make a sound or give some other indication to the user when gas is flowing through the relief valve so that the user can respond by adjusting the oxygen flow into the circuit, adjusting their breathing, or adjusting some other parameter; and filtering, humidifying, dehumidifying, heating, cooling, purifying, or otherwise conditioning the gas in the breathing circuit.

In general, the method of delivering oxygen gas for rebreathing, whether using one of the breathing circuits presented herein or a different rebreathing circuit is such that the flow of oxygen delivered to the breathing circuit is less than the minute ventilation rate but greater or equal to the metabolic oxygen consumption rate of the user.

A typical session of rebreathing with one of the circuits presented herein may involve several different phases of oxygen delivery to the circuit. A first possible phase (pre-fill) is to fill the circuit and counterlung—which may expand to accommodate the additional gas volume—with high concentration oxygen gas before the user begins breathing with the circuit. In this phase, oxygen can be fed into the circuit through the intake port. Any oxygen flow rate will serve to fill the circuit, with higher flow rates filling the circuit more quickly. Oxygen delivered to the circuit during this phase may also flush out other gasses in the circuit prior to use.

It is possible to begin breathing on the circuits described herein without prefilling the circuit by providing a high oxygen flow rate is required to satisfy the user's minute ventilation (volume of gas being inhaled and exhaled) and still provide additional oxygen for filling the circuit.

A typical session of rebreathing may also involve an initial phase (flush mode) during which exhaled breath is vented out of the circuit followed by a phase (maintenance mode) during which the exhaled breath is received back into the circuit. As discussed above, initial venting of exhaled breath clears out the low (or normal) oxygen concentration gas mixture which fills the circuit and the user's lungs before use of the breathing circuit. This ensures that gas inhaled during the flush phase will then consist almost entirely of high concentration oxygen gas fed into the circuit through the intake port. During the flush phase or mode or operation, highly oxygenated gas flow into the circuit will be substantially higher than flow into the circuit during the maintenance phase or mode of operation.

The modes of operation described above may be controlled manually by means of a flow regulator which regulates flow from the oxygen source with a fully adjustable flow setting, or a flow regulator with a limited number of fixed flow settings such as ‘off’, ‘low flow’, and ‘high flow. These phases may also be controlled automatically by means of a demand valve which allows oxygen into the breathing circuit as needed based on pressure changes within the breathing circuit.

The temperature and/or humidity of the gas in the circuit may increase as it passes in and out of the user's lungs. During a rebreathing session it may therefore also be desirable to flush the contents of the circuit and refill it with ‘fresh’ oxygen from the oxygen source by repeating one or more of the phases described above. For the same reason, it may also be beneficial to include a cooling mechanism and/or dehumidifier as part of the circuit.

The circular circuit also comprises an expiratory relief valve on the breathing interface side of the first pathway one-way valve and the second pathway one-way valve which allows gas out of the circuit when the pressure across the expiratory relief valve is higher inside the circuit than outside the circuit by an amount greater than the cracking pressure of the expiratory relief valve.

In some embodiments, the expiratory relief valve has an adjustable cracking pressure that can be set to be more or less than the cracking pressure of the one-way valve in the second pathway. When the cracking pressure of the expiratory relief valve is more than the cracking pressure of the one-way valve in the second pathway, gas will flow through the one-way valve from the breathing interface to the counterlung during exhalation. When the cracking pressure of the expiratory relief valve is less than the cracking pressure of the one-way valve in the second pathway, gas will flow out of the circuit through the expiratory relief valve. One or more of the one-way valves above may comprise a check valve or a solenoid valve.

As described in connection with the linear circuits, the expiratory relief valve with an adjustable cracking pressure in this circular circuit can be a port than can be opened, to set a very low cracking pressure or closed, to set a very high cracking pressure. It can further comprise an additional one-way valve to prevent gas from flowing into the circuit through the port when the port is open.

In any of the embodiments described above, it may be desirable to include one or more of the following additional elements: one or more gas flow sensors to measure gas flow through one or more parts of the circuit; one or more gas sensors (e.g., O2 lambda, etc.) to measure the concentrations of one or more gasses in the circuit; one or more sensors or timers to measure the status or usage of the scrubber and indicate when the scrubber is reaching the end of its effective use; one or more devices added to one or more of the relief valves that will make a sound or give some other indication to the user when gas is flowing through the relief valve; an air filter, humidifier, dehumidifier, heater, cooler, purifier, or other device for conditioning the gas in the circuit. Additional ports on one or more components of the breathing circuit may also be needed to enable coupling of such additional elements to the circuit.

In any of the embodiments described above it may also be desirable to design the circuit comprising one or more of the following features: the gas counterlung can be removed from the circuit and reattached or replaced; the gas reservoir and carbon dioxide scrubber are combined in a single counterlung-and-scrubber module which can be separated from the rest of the circuit and reconnected or replaced; the size of the carbon dioxide scrubber is matched to the amount of CO2 expected to pass through the scrubber during the expected time of use of the circuit during a single session of use.

The efficient method of therapeutically delivering gas with a higher than normal concentration of oxygen disclosed in the present invention can be performed using any of the breathing circuits described above. The rebreathing circuits presented in the present disclosure have advantages over other circuits known in the art as already discussed (more efficient use of oxygen, higher oxygen concentrations, faster increase in oxygen concentration, smaller size, etc.). Their novelty delivers breathable high concentration oxygen gas and achieves the desired result of inducing vasoconstriction in response to elevated levels of blood oxygen. This response may be beneficial in treating neurovascular conditions s including headache, cluster headache, and migraine headache. The inventors of the present application recognize that the use of a rebreathing circuit in the treatment of neurovascular conditions has not been previously disclosed.

The method of delivering and rebreathing gasses with the rebreathing circuits described in this disclosure might also be used for breathing or rebreathing of other gas mixtures, or in other applications besides treating ischemic conditions. When the gas mixture being delivered and rebreathed contains oxygen concentrations higher than normal air, the range of concentrations can be anywhere from slightly or moderately higher concentration than normal air up to substantially pure oxygen. However, the benefits of rebreathing the gas mixture and recycling the unused oxygen increase as the desired oxygen concentration increases.

More specifically, the method of oxygen delivery using the rebreathing circuits described above comprises delivering oxygen to a breathing circuit through a gas intake port for inhalation through a breathing interface such as a mask or mouthpiece; receiving exhaled gas back into the breathing circuit through the breathing interface; removing carbon dioxide from the gas in the breathing circuit using a carbon dioxide scrubber; and storing oxygen and/or exhaled gas in a gas counterlung in the breathing circuit; and, all in such a way that gas moving from the breathing interface to the counterlung follows substantially the same pathway as gas moving from the reservoir to the breathing interface.

The method may optionally further comprise one or more of: allowing air into the circuit through an inspiratory relief valve when inhalation draws more gas than available inside the breathing circuit; allowing air out of the circuit through an expiratory relief valve when the volume and/or pressure of gas inside the breathing circuit is too high; switching between a first state in which the exhaled gas received back into the circuit is directed into the counterlung and a second state in which the exhaled gas is vented out of the breathing circuit; measuring the pressure, flow, and/or concentration of gasses in the circuit; measuring and indicating the status of the scrubber and its remaining scrubbing capacity; adding to one or more of the relief valves a device that will make a sound or give some other indication to the user when gas is flowing through the relief valve so that the user can respond by adjusting the oxygen flow into the circuit, adjusting their breathing, or adjusting some other parameter; and filtering, humidifying, dehumidifying, heating, cooling, purifying, or otherwise conditioning the gas in the breathing circuit.

In general, the method of delivering oxygen gas for rebreathing, whether using one of the breathing circuits presented in this patent is such that the volume of oxygen delivered to the breathing circuit is less than the minute ventilation rate but greater or equal to the metabolic oxygen consumption rate of the user.

A typical session of rebreathing with one of the circuits presented in this disclosure may involve several different phases of oxygen delivery to the circuit. A first possible phase is to fill the circuit and counterlung—which may expand to accommodate the additional gas volume—with high concentration oxygen gas before the user begins breathing with the circuit. In this phase, oxygen can be fed into the circuit through the intake port. Any oxygen flow rate will serve to fill the circuit, with higher flow rates filling the circuit more quickly. Oxygen delivered to the circuit during this phase may also flush out other gasses in the circuit prior to use.

A second possible phase is filling of the circuit while the user has begun breathing with the circuit. In this phase, a high oxygen flow rate is required to satisfy the user's minute ventilation (volume of gas being inhaled and exhaled) and still provide additional oxygen for filling the circuit.

A third possible phase is adding oxygen after the circuit has been filled in order to replace oxygen consumed by the user and/or which has escaped from the circuit, so that the volume and/or concentration of oxygen in the circuit is maintained. This phase requires a much lower flow rate.

A typical session of rebreathing may also involve an initial phase during which exhaled breath is vented out of the circuit followed by a phase during which the exhaled breath is received back into the circuit. As discussed above, initial venting of exhaled breath clears out the low (or normal) oxygen concentration gas mixture which fills the circuit and the user's lungs before use of the breathing circuit, and therefore enables much higher oxygen concentrations in the gas mixture stored in the circuit, which will then consist almost entirely of high concentration oxygen gas fed into the circuit through the intake port.

The temperature and/or humidity of the gas in the circuit may increase as it passes in and out of the user's lungs. During a rebreathing session it may therefore also be desirable to flush the contents of the circuit and refill it with ‘fresh’ oxygen from the oxygen source by repeating one or more of the phases described above. For the same reason, it may also be beneficial to include a cooling mechanism and/or dehumidifier as part of the circuit.

In summary, the rebreathing circuits and methods described herein are especially useful for high concentration oxygen therapy because in such scenarios large volumes of oxygen are inhaled and exhaled without being consumed. Only a small amount of inhaled oxygen is transferred into the bloodstream and replaced by carbon dioxide to make up the exhaled breath. Therefore, in conventional approaches most of the inhaled oxygen ends up being exhaled into the surrounding air and lost without being consumed by the patient. In this invention, the exhaled oxygen is reused and recycled instead of being released into the surrounding air, which significantly reduces the amount of wasted oxygen and the total volume of oxygen required compared to the conventional approach of supplying enough oxygen with each breath to satisfy full ventilation.

Having thus described several aspects and embodiments of the technology of this application, it is to be appreciated that various alterations, modifications, and improvements will readily occur to those of ordinary skill in the art. Such alterations, modifications, and improvements are intended to be within the spirit and scope of the technology described in the application. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein.

The present invention should therefore not be considered limited to the particular embodiments described above. Various modifications, equivalent processes, as well as numerous structures to which the present invention may be applicable, will be readily apparent to those skilled in the art to which the present invention is directed upon review of the present disclosure.

What is claimed is:

1. A method for treating pain in a patient, comprising:
capturing exhaled gas from the patient, and scrubbing at least part of the exhaled gas using a carbon dioxide scrubber; and
delivering to the patient a mixture of scrubbed exhaled gas as well as highly oxygenated gas from a source of highly oxygenated gas, wherein said highly oxygenated gas is provided from said source at a rate below the patient's minute ventilation rate.

2. A method for treating pain in a patient, using a breathing apparatus, the method comprising:
coupling the breathing apparatus to a source of highly oxygenated gas, said highly oxygenated gas having an oxygen concentration greater than that of a naturally occurring environment;
providing a breathing interface of said breathing apparatus to the patient to inhale from and to exhale into;
in a first (flushing) mode of operation: (a) discharging exhaled gas from the patient to said environment, and (b) providing the patient for inhalation said highly oxygenated gas, from said source, via said breathing apparatus; and
in a second (maintenance) mode of operation: (a) receiving the patient's exhaled gas in said breathing apparatus, (b) scrubbing at least part of said patient's exhaled gas with a carbon dioxide scrubber in said breathing apparatus, and (c) providing the patient for inhalation at least part of the scrubbed exhaled gas.

3. The method of claim 2, said method applied to treat a headache in said patient, and continued until said patient's headache symptoms are acceptably reduced.

4. The method of claim 3, said method applied to treat a cluster headache in said patient, and continued until said patient's cluster headache symptoms are acceptably reduced.

5. A method for treating pain in a patient, using a breathing apparatus that the patient inhales from and exhales to, the method comprising:
capturing exhaled gas from the patient, and scrubbing at least part of the exhaled gas using a carbon dioxide scrubber in said breathing apparatus; and
delivering to the patient a mixture of scrubbed exhaled gas as well as highly oxygenated gas from a gas source, said highly oxygenated gas having a concentration of oxygen greater than an atmospheric concentration of oxygen, and said highly oxygenated gas being delivered at a flow rate less than a minute ventilation rate of the patient.

6. The method of claim 5, further comprising operation in a flush mode by opening a discharge port of said breathing apparatus and wherein a majority of the captured exhaled gas from the patient is discharged from the breathing apparatus through said opened discharge port.

7. The method of claim 6, wherein said majority of the captured exhaled gas comprises substantially all of the captured exhaled gas.

8. The method of claim 5, further comprising operation in a maintenance mode wherein a discharge port of said breathing apparatus is closed and wherein a majority of the captured exhaled gas from the patient is retained within the breathing apparatus and cannot exit the apparatus through said closed discharge port.

9. The method of claim 8, wherein said majority of the captured exhaled gas comprises substantially all of the captured exhaled gas.

10. The method of claim 5, further comprising actuating a switch to switch operation between a flush mode of operation wherein a discharge port of said breathing apparatus is opened and substantially all of the captured exhaled gas from the patient is discharged from the breathing apparatus through said opened discharge port, and a maintenance mode of operation wherein said discharge port is closed and substantially all of the captured exhaled gas from the patient is retained in the breathing apparatus and cannot exit the apparatus through said discharge port.

11. The method of claim 5, further comprising continuing to deliver said mixture of gases to the patient until a desired reduction in said patient's pain has been achieved.

12. The method of claim 10, further comprising continuing to deliver said mixture of gases to the patient until a desired reduction in said patient's headache has been achieved.

13. The method of claim 12, further comprising continuing to deliver said mixture of gases to the patient until a desired reduction in said patient's cluster headache has been achieved.

14. A breathing apparatus, comprising:
a breathing interface from which a patient inhales and into which the patient exhales;
a gas source containing a highly oxygenated gas having a greater concentration of oxygen than an atmospheric concentration of oxygen;
a supply gas inlet, placing said apparatus in fluid communication with said gas source, and providing a flow of said highly oxygenated gas to the patient;
a gas flow regulator that regulates a rate of supply of said highly oxygenated gas such that said rate of supply is at least sometimes less than a minute ventilation rate of said patient during use; and
a carbon dioxide scrubber that receives exhaled gas from the patient;
wherein the highly oxygenated gas provided through the gas inlet, and the exhaled gas scrubbed by the carbon dioxide scrubber are provided through said breathing apparatus, via the breathing interface, to the patient inhaling therefrom.

15. The apparatus of claim 14, further comprising a switchable discharge port configured and arranged to be opened during a flush mode of operation so as to discharge exhaled gas out of said breathing apparatus, and to be closed during a maintenance mode of operation so as to capture the exhaled gas within the breathing apparatus.

16. The apparatus of claim 15, said switchable discharge port comprising an electromechanically controllable valve or switch that causes said discharge port to be operated in an opened or in a closed mode.

17. The apparatus of claim 16, wherein said electromechanically controllable valve or switch is manually operated.

18. The apparatus of claim 16, wherein said electromechanically controllable valve or switch is machine-operated based on an input signal determining a mode of operation thereof.

19. The apparatus of claim 14, said gas flow regulator comprising a demand valve that opens on demand according to said patient's breathing requirements.

20. The apparatus of claim 14, said gas flow regulator comprising a manual or automated gas flow rate control valve.

21. A method of easing pain in a patient using a breathing apparatus coupled to a highly oxygenated gas source and having a gas reservoir and a carbon dioxide scrubber, the method comprising:
capturing exhaled gas from said patient in said reservoir;
removing carbon dioxide from said exhaled gas using said carbon dioxide scrubber;
providing the patient, for inhalation through the breathing apparatus, at least a portion of the captured exhaled gas and at least a portion of the highly oxygenated gas from the source of highly oxygenated gas; and
continuing to provide the aforementioned gases to the patient, for inhalation, until a desired reduction in said pain is achieved.

22. The method of claim 21, further comprising regulating a flow rate of said highly oxygenated gas from the source.

23. The method of claim 22, further comprising limiting said flow rate to a value less than a minute ventilation rate of said patient.
